(19) United States
(12) Patent Application Publication
(10) Pub. No.: US 2025/0354784 A1
(43) Pub. Date: Nov. 20, 2025

(54) RESTRAINT METHODS

(71) Applicant: Gracie Global, LLC, Torrance, CA (US)

(72) Inventor: Rener Gracie, Torrance, CA (US)

(21) Appl. No.: 19/208,043

(22) Filed: May 14, 2025

Related U.S. Application Data
(60) Provisional application No. 63/647,962, filed on May 15, 2024.
(60) Provisional application No. 63/698,377, filed on Sep. 24, 2024.

(51) Int. Cl. F41H 13/00

(52) U.S. Cl. CPC F41H 13/00

(57) ABSTRACT

A method for restraint of a subject in a supine position is described. The method comprises pulling a first arm of the subject over a shoulder of a second arm of the subject to achieve a cross-over arms position. The method further comprises rolling the subject from the supine position to a lateral position while preserving the cross-over arms position. The method further comprises pulling a first leg of the subject over a second leg of the subject to achieve a cross-over legs position. The method further comprises preserving the cross-over legs position of the subject by securely restraining at least the first leg of the subject. The cross-over arms position and the cross-over legs position are preserved simultaneously for restraint of the subject. Further, methods for restraint of a subject in a prone position and seated position are also provided.

### CROSS-REFERENCE TO RELATED APPLICATIONS

This application claims the benefit of U.S. Provisional Patent Application Ser. No. 63/647,962 filed on May 15, 2024, and U.S. Provisional Patent Application Ser. No. 63/698,377 filed on Sep. 24, 2024, both of which are incorporated by reference herein in their entirety.

### FIELD OF THE INVENTION

The present invention relates generally to restraining individuals, and more particularly to methods for restraint of an individual in a manner that minimizes the risk of injury, panic, asphyxiation, and/or strangulation.

### BACKGROUND OF THE INVENTION

Many human service and law enforcement agencies are responsible for the care and supervision of individuals who need to be restrained, such as aggressive, suicidal, and emotionally disturbed individuals. The staff and officers in these agencies regularly engage in physical restraint techniques.

In many human service and law enforcement settings, staff members frequently face situations where they must physically restrain emotionally disturbed persons (EDPs) to prevent harm. Traditional restraint methods, particularly prone restraints, have been associated with significant risks, including compression asphyxia and strangulation, which can result in severe injury or even death.

Historically, prone restraint has been a common method of restraining an individual. However, this method has several significant flaws associated with the tragic deaths of civilians and law enforcement officers alike.

Recognizing the dangers inherent in prone restraint, once an individual is restrained, the individual is preferably moved out of prone restraint into a lateral recumbent position or seated position. Unfortunately, the process of applying a restraining device, such as handcuffs, can take several minutes, during which the safety of all parties is at increased risk. Up to this point, there were no feasible alternatives to the prone restraint position when it came to applying a restraining device, such as handcuffs. Therefore, reducing risks associated with the position involved an attempt to minimize the amount of time that was spent in the prone position.

One perceived benefit of prone restraint is that it positions the individual with their arms beneath their body, making it harder for them to launch an attack. However, this advantage is offset by the significant challenge of extracting the individual's hands for handcuff application once they are buried under the body.

The conventional ways of restraint require one or more individuals applying various means of force against a single subject. Such force has been associated to death of the subject due, for example, positional asphyxiation, compression asphyxiation, or strangulation when the subject is, for example, restrained in a supine or prone position and forced to carry the weight of the restraining individuals directly on their diaphragm, neck, or arteries.

Other common ways to hold an individual include a basket hold, leg-lace, figure-four leg control, etc. In the basket hold, a first restrainer crosses the subject's arms, and the wrists are controlled. The subject can be pulled to the ground on their side and held the basket hold while a second restrainer can lay on the upper thighs of the subject. However, the basket hold is ineffective because the subject can use the force of their hips to get up to the knees and try to escape. Such a scenario would generally lead to escalation of force.

Figure-four control and leg-laces generally pertain to law enforcement encounters in which the subject is pronated, and the legs of the subject are crossed. Typically, the tibia of one leg goes behind the knee or tibia of the other leg and then the restrainer attempts to secure this position. While such configuration may help to immobilize the subject, the subject can still use their hands to push up from the ground which generally requires more body weight to be placed on the upper torso, by the second restrainer, to maintain the position of control. Furthermore, because the subject is in prone position when pressure is applied on their upper torso by a second restrainer, the risk of lung compression remains. Moreover, the restrainers do not have visual of the subject's face and cannot detect any medical condition.

Accordingly, there is an established need for a solution to the problems mentioned above. For instance, there is an established need for systems and methods that ensure the restrained individual remains in a medically desirable lateral position. Further, there is an established need for systems and methods that significantly reduce any potential risks of asphyxia, strangulation, and injury while facilitating communication, de-escalation, and subsequent procedures.

### SUMMARY OF THE INVENTION

In one aspect, a method of restraining a human subject includes manipulating the subject into a restrained position. The restrained position includes a portion of a first arm of the subject over a shoulder of a second arm of the subject to achieve a restrained cross-over arms position and a portion of a first leg of the subject over a second leg of the subject to achieve a restrained cross-over legs position, wherein the first arm and the first leg are on opposite sides of a body of the subject. The method further includes manipulating the subject to apply a restraining device to the first and second arms of the subject to restrain the first and second arms and applying the restraining device to at least one of the first and second arms of the subject.

In another aspect, the present invention relates to a method for lateral restraint of a subject by a first restrainer and a second restrainer. The method includes pulling a first arm of the subject over a shoulder of a second arm of the subject, to achieve a cross-over arms position. The method further includes preserving the cross-over arms position by securely restraining at least the first arm of the subject. The method further includes pulling a first leg of the subject over a second leg of the subject, to achieve a cross-over legs position. The method further includes preserving the cross-over legs position by securely restraining at least the first leg of the subject. The cross-over arms position and the cross-over legs position are preserved in tandem to secure the subject in the lateral restraint.

In an embodiment, the cross-over arms position may be preserved by the first restrainer.

In an embodiment, the cross-over legs position may be preserved by the second restrainer.

In an embodiment, the method may include securely restraining the second arm of the subject.

In an embodiment, preserving the cross-over legs position may include securely restraining the first leg of the subject by means of one or more of armpits, ribs area, knee area, or by hands of one of the first restrainer and the second restrainer.

In an embodiment, preserving the cross-over arms position may include securely restraining the at least the first arm via the wrist.

In an embodiment, when the subject is in a supine position, the method may include rolling the subject from the supine position to a lateral position.

In an embodiment, when the subject is in the lateral position on the left side of the subject, the first arm may be the right arm while the first leg may be the left leg.

In an embodiment, when the subject is in the lateral position on the right side, the first arm may be the left arm while the first leg may be the right leg.

In an embodiment, when the subject is in a prone position, the method may include shifting the subject to the lateral position.

In an embodiment, shifting the subject from the prone position to the lateral position may include bending the first leg at the knee to raise the tibia of the first leg; applying a pushing force on the tibia of the first leg to position the tibia over the femur and the hip of the first leg; and applying a lateral force on the tibia of the first leg to rotate the tibia towards the ground, thereby causing rotation of the hips and torso of the subject and shifting the subject to the lateral position.

In an embodiment, the method may further include transitioning from the lateral restraint to a restraining device application position, such as a handcuffing position, in which the subject is handcuffed.

In an embodiment, transitioning from the lateral restraint to the restraining device application position may include lifting the head of the subject; pulling the second arm of the subject behind the back of the subject while preserving the cross-over arms position and the cross-over legs position; handing over control of the first arm from the first restrainer to the second restrainer; applying a restraining device on the second arm of the subject while preserving the cross-over legs position; and pulling the first arm behind the subject and applying the restraining device, such as handcuffs, on the first arm of the subject while preserving the cross-over legs position.

In an embodiment, the method may further include transitioning from the lateral restraint to a four-point restraint position in which the subject is restrained on all four limbs.

In an embodiment, transitioning from the lateral restraint to the four-point restraint position may include restraining the second arm and second leg prior to the first arm and first leg.

In an embodiment, transitioning from the lateral restraint to the four-point restraint position may include restraining of the second arm, followed by the second leg, followed by the first leg, and followed by the first arm.

In a further embodiment, a method of restraining a human subject includes manipulating the subject into a restrained position. The restrained position includes a portion of a first arm of the subject over a shoulder of a second arm of the subject to achieve a restrained cross-over arms position and a portion of a first leg of the subject over a second leg of the subject to achieve a restrained cross-over legs position, wherein the first arm and the first leg are on opposite sides of a body of the subject.

In another aspect, a method for restraint of a subject in a supine position is described. The method includes pulling a first arm of the subject over a shoulder of a second arm of the subject to achieve a cross-over arms position. The method further includes rolling the subject from the supine position to a lateral position while preserving the cross-over arms position. The method further includes pulling a first leg of the subject over a second leg of the subject to achieve a cross-over legs position. The method further includes preserving the cross-over legs position of the subject by securely restraining at least the first leg of the subject. The cross-over arms position and the cross-over legs position are preserved simultaneously for restraint of the subject.

In an embodiment, the cross-over arms position may be preserved by a first restrainer and the cross-over legs position may be preserved by a second restrainer.

In an embodiment, preserving the cross-over arms position may include securely restraining at least the first arm of the subject by the first restrainer.

In an embodiment, the method may further include securely restraining the second arm of the subject by the first restrainer.

In an embodiment, preserving the cross-over legs position may include securely restraining the first leg of the subject by means of one or more of armpits, ribs area, knee area, or by hands of the second restrainer.

In an embodiment, preserving the cross-over legs position may include raising the legs of the subject by the second restrainer; extending arms of the second restrainer under the raised legs of the subject; and securing hands of the second restrainer together to secure the first leg and the second leg in the cross-over legs position.

In an embodiment, in the cross-over legs position, a tibia of the first leg may be crossed over a femur of the second leg.

In an embodiment, the method may further include transitioning the subject to a restraining device application position by handing over at least partial control of the second arm from the first restrainer to the second restrainer; lifting the head of the subject; pulling the second arm of the subject behind the back of the subject while preserving at least the cross-over legs position; handing over control of the first arm from the first restrainer to the second restrainer; applying a restraining device on the second arm of the subject while preserving the cross-over legs position; pushing the first arm of the subject behind the back of the subject; and applying handcuffs on the first arm of the subject.

In an embodiment, when the subject is in the lateral position on its left side, the first arm may be the right arm of the subject while the first leg may be the left leg of the subject.

In an embodiment, when the subject is in the lateral position on its right side, the first arm may be the left arm of the subject while the first leg may be the right leg of the subject.

In an embodiment, the method may further include transitioning the subject to a four-point restraint position by extending the second arm of the subject and attaching a respective physical restraint to the second arm; extending the second leg of the subject and attaching a respective physical restraint to the second leg; extending the first leg of the subject and attaching a respective physical restraint to the first leg; and extending the first arm of the subject and attaching a respective physical restraint to the first arm.

In yet another aspect, a method for restraint of a subject in a prone position includes bending a first leg of the subject at the knee so as to raise a tibia of the first leg; applying a pushing force on the tibia of the first leg to position the tibia over a femur and a hip of the first leg; rotating the tibia of the first leg towards the ground by applying a lateral force on the first leg, thereby causing rotation of the hips and torso of the subject and shifting the subject to a lateral position; pulling a first arm of the subject over a shoulder of a second arm of the subject to achieve a cross-over arms position; preserving the cross-over arms position of the subject by securely restraining at least the first arm of the subject; pulling the first leg of the subject over a second leg of the subject to achieve a cross-over legs position; and preserving the cross-over legs position of the subject by securely restraining at least the first leg of the subject. The cross-over arms position and the cross-over legs position are preserved simultaneously for restraint of the subject. The method may further include applying a restraining device to the first and second arms of the subject to restrain the first and second arms.

In an embodiment, the cross-over arms position may be preserved by a first restrainer and the cross-over legs position may be preserved by a second restrainer.

In an embodiment, when the subject is in the lateral position on its left side, the first arm may be the right arm while the first leg may be the left leg.

In an embodiment, when the subject is in the lateral position on its right side, the first arm may be the left arm while the first leg may be the right leg.

In an embodiment, the method may further include transitioning the subject to a restraining device application position by handing over at least partial control of the second arm from the first restrainer to the second restrainer; lifting the head of the subject; pulling the second arm of the subject behind the back of the subject while preserving at least the cross-over legs position; handing over control of the first arm from the first restrainer to the second restrainer; applying a restraining device on the second arm of the subject while preserving the cross-over legs position; pushing the first arm of the subject behind the back of the subject; and applying the restraining device on the first arm of the subject.

In an embodiment, the method may further include transitioning the subject to a four-point restraint position in which all four limbs of the subject are restrained, wherein transitioning from the lateral restraint to the four-point restraint position may include restraining the second arm and second leg of the subject prior to the first arm and first leg of the subject.

In an embodiment, transitioning the subject to the four-point restraint position may include sequentially restraining the second arm, followed by the second leg, followed by the first leg, and followed by the first arm of the subject.

In an embodiment, when the subject is shifted in the lateral position on its left side, the tibia of the first leg may be rotated in an anti-clockwise direction.

In an embodiment, when the subject is shifted in the lateral position on its right side, the tibia of the first leg may be rotated in a clockwise direction.

In yet another aspect, a method for restraint of a subject in a seated position includes directing a first arm of the subject over a shoulder of a second arm of the subject to achieve a cross-over arms position; preserving the cross-over arms position by securely restraining the first arm and the second arm of the subject; crossing a first leg of the subject over a knee of a second leg of the subject to achieve a cross-over legs position; preserving the cross-over legs position of the subject. The cross-over arms position and the cross-over legs position are preserved in tandem to restraint the subject in the seated position.

In an embodiment, the method may further include raising the second arm of the subject while preserving the cross-over legs position; extending the first arm underneath the raised second arm of the subject; and lowering the raised second arm of the subject so as to secure the first arm with the second arm.

In an embodiment, the cross-over arms position may be preserved by a first restrainer and the cross-over legs position may be preserved by a second restrainer.

In an embodiment, when the first and second restrainers are on a left side of subject, the first arm may be the left arm of the subject while the first leg may be the right leg of the subject.

In an embodiment, when the first and second restrainers are on a right side of subject, the first arm may be the right arm of the subject while the first leg may be the left leg of the subject.

These and other objects, features, and advantages of the present invention will become more readily apparent from the attached drawings and the detailed description of the embodiments and examples, which follow.

### BRIEF DESCRIPTION OF THE DRAWINGS

The preferred embodiments of the invention will hereinafter be described in conjunction with the appended drawings provided to illustrate and not to limit the invention, where like designations denote like elements, and in which:

FIGS. 1A, 1B, and 2-4 depict sequential steps of an exemplary method for restraining a subject when the subject is initially in the supine position, in accordance with one embodiment of the present specification;

FIGS. 5-12 depict sequential steps of transitioning from a lateral restraint position to a restraining device application position, in accordance with one embodiment of the present specification;

FIGS. 13-20 depict sequential steps of an exemplary method for restraining a subject when the subject is initially in the prone position, in accordance with one embodiment of the present specification;

FIGS. 21-25 depict sequential steps of an exemplary method of transitioning from a lateral restraint position to a four-point restraint position, in accordance with another embodiment of the present specification; and

FIGS. 26 and 27 depict sequential steps of an exemplary method for restraining a subject when the subject is in a seated position, in accordance with one embodiment of the present specification;

Like reference numerals refer to like parts throughout the several views of the drawings.

### DETAILED DESCRIPTION

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other embodiments. All of the embodiments described below are exemplary embodiments provided to enable persons skilled in the art to make or use the embodiments of the disclosure and are not intended to limit the scope of the disclosure, which is defined by the claims. For purposes of description herein, the terms “upper”, “lower”, “left”, “rear”, “right”, “front”, “vertical”, “horizontal”, and derivatives thereof shall relate to the invention as oriented in the drawings. Furthermore, there is no intention to be bound by any expressed or implied theory presented in the preceding technical field, background, brief summary or the following detailed description. It is also to be understood that the specific devices and processes illustrated in the attached drawings and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise.

In the following description, certain specific details are set forth in order to provide a thorough understanding of various disclosed embodiments. However, one skilled in the relevant art will recognize that embodiments may be practiced without one or more of these specific details, or with other methods, components, materials, and the like. In other instances, well-known elements associated with restraining methods have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is, as “including, but not limited to.”

As used in this specification and the appended claims, the singular forms “a,” “an,” and “the” include plural referents unless the content clearly dictates otherwise, and the vice versa. It should also be noted that the term “or” is generally employed in its broadest sense, that is, as meaning “and/or” unless the content clearly dictates otherwise.

The headings and Abstract of the Disclosure provided herein are for convenience only and do not interpret the scope or meaning of the embodiments.

Embodiments of the methods described herein apply to the restraint of an individual, such as restraining aggressive, suicidal, or emotionally disturbed persons (EDPs) in a manner that minimizes the risk of asphyxiation and/or strangulation. The methods as described herein are applicable across various fields, including law enforcement, healthcare, first responders, security, and education, where staff members are required to safely control individuals who pose a danger to themselves or others.

The method generally includes two people restraining a third person who is, for example, on the ground, on an elevated surface (such as a stretcher or hospital bed), or in a chair (such as an airplane seat) for extended periods while significantly reducing the dangers of positional asphyxia, compression asphyxia or strangulation. With this method, the third person can be restrained in a way that does not impede breathing or vascular structures. The method involves positioning and control of the third person's limbs to maintain a restraint position, thereby preventing escape and ensuring safety for all parties involved.

For ease of reference, the method will be described with reference to a first person and a second person restraining a subject. The subject refers to the person being restrained. The first person (or the first restrainer) refers to the person restraining the upper body of the subject. The second person (or the second restrainer) refers to the person retraining the lower body of the subject.

In one aspect, the subject may be in a supine position in which the subject is lying horizontally on the back with the spine facing the ground and the face and torso facing upwards. The methods for restraining the subject in the supine position will now be described.

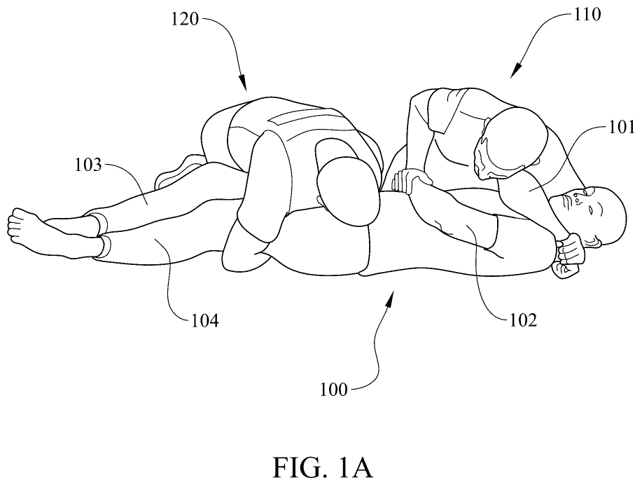

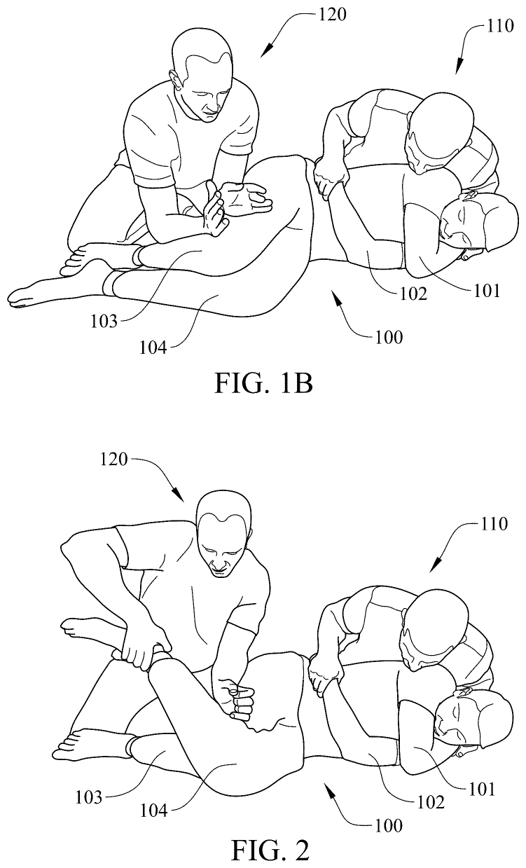

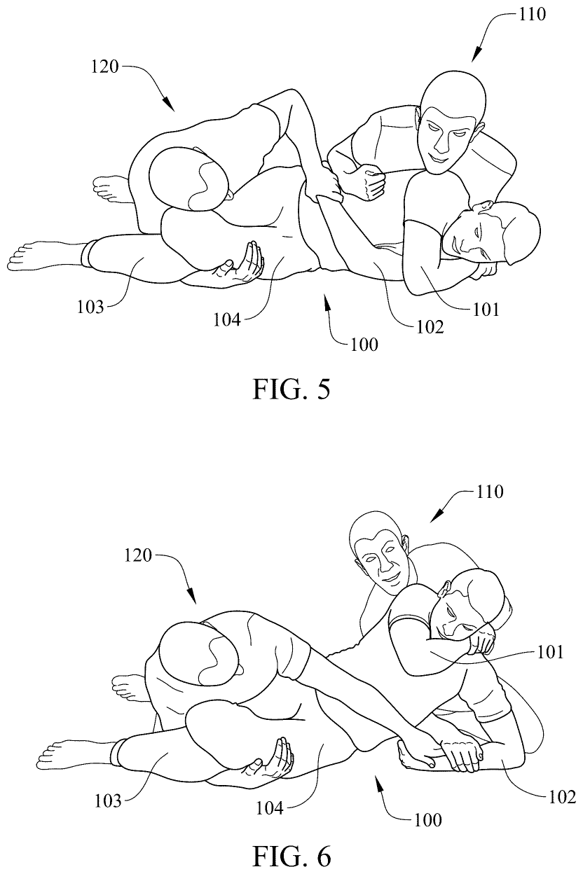

FIGS. 1-4 depict sequential steps of an exemplary restraining method for laterally restraining a subject 100 when the subject 100 is initially in the supine position. FIGS. 5-12 depict sequential steps of transitioning from a lateral restraint position to a restraining device application position. The method may be performed by the first person 110 and the second person 120. The following description describes the restraining method from a perspective of the first person 110 and the second person 120 positioned on the subject 100's right side. The description equally applies to a perspective of the first person 110 and the second person 120 positioned on the subject 100's left side with specific actions being performed on the opposite limbs described below. Note, any restraining device suitable to restrain the arms of a human subject can be used as an integral part of the restraining method described herein. Handcuffs are one example of such a suitable restraining device. Other restraining devices include, zip ties, rope, cord, belts, string, shoelaces, ctc. Accordingly, references below to “handcuffs” and “handcuffing position” herein equally applies to other restraining devices suitable to restrain the arms of a human subject. The phrase “lateral restraint position” refers to a position where the subject is on a side of the subject.

Initially, the subject 100 may be in the supine position as depicted in FIG. 1A. The subject 100 may be on the ground, on a gurney, a hospital bed, an elevated surface, and the like. As depicted in FIG. 1A, the first person 110 and the second person 120 may position themselves on one side of the subject 100, ensuring full access to the body of the subject 100. In one example, the first person 110 may hold the arms of the subject 100 while the second person 120 may grip the legs of the subject 100. The subject 100 may include a right arm 101 and a left arm 102. The first person 110 may secure the upper body of the subject 100 by crossing the right arm 101 over the shoulder of the left arm 102, as depicted in FIG. 1A. The first person 110 may hold both the right arm 101 and the left arm 102 of the subject 100, for instance, via the wrists of the subject 100. The first person 110 secures the cross-over arms position by holding one or both of the subject's wrists, ensuring that the subject's arms are crossed in front of their body. In the foregoing embodiment with the first person 110 and the second person 120 positioned on the subject 100's right side, the right arm 101 may be referred to as the first arm while the left arm 102 may be referred to as the second arm. As previously mentioned, the restraining method can also be performed in an opposite side position with the first person 110 and the second person 120 positioned on the subject 100's left side. In this opposite position, the right arm 101 may be referred to as the second arm while the left arm 102 may be referred to as the first arm.

As seen in FIG. 1B, the first person 110 and/or the second person 120 may roll the body of the subject 100 laterally so that the subject 100 faces away from the first person 110 and the second person 120. The second person 120 may grip or apply a force on the lower body of the subject 100. The first person 110 may secure the upper body of the subject 100 with crossing-over of the arms of the subject 100. As seen in FIG. 2, the first person 110 may continue holding the right arm 101 and the left arm 102 of the subject 100 in the cross-over arms position thereof. The second person 120 may secure the lower body of the subject 100, in particular, a right leg 103 and a left leg 104 of the subject 100. The second person 120 may initiate pulling of the left leg 104 over the right leg 103 so as to cross the left leg 104 over the right leg 103. In an embodiment, the tibia of the left leg 104 is crossed over the femur of the right leg 103. In the foregoing embodiment with the first person 110 and the second person 120 positioned on the subject 100's right side, the right leg 103 may be referred to as the second leg while the left leg 104 may be referred to as the first leg. As previously mentioned, the restraining method can also be performed in an opposite side position with the first person 110 and the second person 120 positioned on the subject 100's left side. In this opposite position, the right leg 103 may be referred to as the first leg while the left leg 104 may be referred to as the second leg.

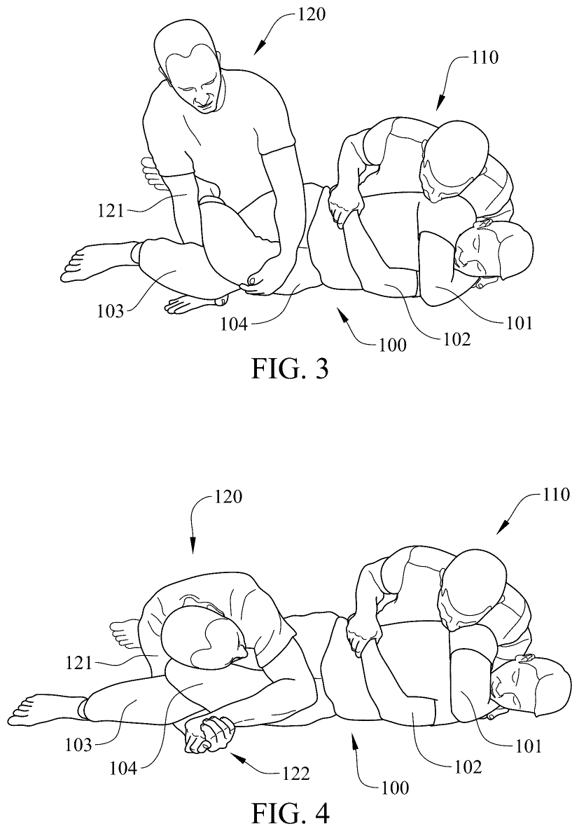

As seen in FIG. 3, once the left leg 104 is over the right leg 103, the second person 120 may initiate securing of the cross-over leg position of the subject 100. In one embodiment, the second person 120 may insert one of his arms, marked with numeral 121, between the left leg 104 and the right leg 103. In particular, when the left leg 104 is over the right leg 103, a space is formed between the feet of the subject 100. The second person 120 may extend the arm 121 in the space. This way, the left leg 104 is secured by the second person 120 between the arm 121 and the torso of the second person 120.

As seen in FIG. 4, the second person 120 may secure the cross-over leg position of the subject 100. The cross-over leg position being secured refers to preserving the position of the tibia of the left leg 104 crossed over the femur of the right leg 103 and preventing the subject 100 from freeing the right leg 103 and the left leg 104 from the cross-over legs position.

In an embodiment, the second person 120 may raise the legs of the subject 100 and extend the arm 121 under the legs of the subject 100. The second person 120 may then securely hold and secure their hands 122 to secure the left leg 104 and the right leg 103 in the cross-over leg position in which the lower body of the subject 100 is restricted from movement. Simultaneously, the first person 110 may continue holding the right arm 101 and the left arm 102 of the subject 100 in the cross-over arms position.

In the illustrated embodiment, the left leg 104 is secured by the second person 120 between the arm 121 and the torso of the second person 120, i.e., using the armpit of the second person 120. In another embodiment, the second person 120 may grip the ankle and foot of the left leg 104 to secure the cross-over legs position. In another embodiment, the second person may hold the ankle and foot of the left leg 104 with his/her own leg, such as by using the knees in a bent position. In another embodiment, the second person may hold the ankle and foot of the left leg 104 with his/her rib area.

The cross-over arms position along with the cross-over legs position of the subject may be referred to as a lateral restraint position of the subject 100. The cross-over arms position simultaneous to the cross-over legs position of the subject prevents the subject from rolling away from the first person 110 and the second person 120, such as, by going to their knees or by turning to a prone position. Further, the cross-over arms position simultaneous to the cross-over legs position of the subject prevents the subject from rolling towards the first person 110 and the second person 120 since a controlled pressure is applied by both the first person 110 and the second person 120 on the subject 100. The lateral restraint position thus inhibits the subject's ability to rotate in either direction, thereby preventing the subject's escape. Moreover, since the lateral restraint position is a medically safe position, and the method of restraint can be used for extended periods of time while minimizing any risk of compression asphyxia or strangulation.

Further, the method of lateral restraint as described herein can be used to transition into the application of restraint devices, such as handcuffs or other devices. As previously set forth, as used herein, the term ‘handcuffing transition’ and ‘handcuff position’ refers to application to any type of restraint devices including but not limited to handcuffs and other restraining devices. The handcuffing transition can be performed without ever relying on prone controls, thereby reducing any risk of asphyxiation or strangulation. The handcuffing transition may be required in cases of law enforcement. The handcuffing transition can be performed a particular time period after the subject 100 has been in the lateral restraint position. For instance, the subject 100 may get tired of trying to escape from the lateral restraint and thereafter the handcuffing transition can be initiated by the first person 110 and the second person 120.

As seen in FIG. 5, the first person 110 may hand over control of the left arm 102 of the subject 100 to the second person 120. The second person 120 may grab the left arm 102 of the subject 100 and the first person 110 may release grip on the left arm 102. In an embodiment, the second person 120 may grab the left arm 102 via the wrist of the subject 100. The first person 110 continues holding the right arm 101 of the subject 100 over the shoulder of the left arm 102 while the second person 120 continues holding the legs of the subject 100.

As seen in FIG. 6, the first person 110 may lift the head of the subject 100. For instance, the first person 110 may apply lifting force taking support from his/her knees or his/her feet. Immediately, the second person 120 may push the left arm 102 of the subject 100 so as to be positioned approximately below the lifted head of the subject 100. For instance, if the subject 100 is on ground, the second person 120 may push the left arm 102 to post the subject's elbow on the ground below the lifted head of the subject 100. Thereafter, the first person 110 may again grip the left arm 102 of the subject 100. In an embodiment, the first person 110 may extend his/her arm under the lifted head and between the chest and left arm of the subject 100, as depicted. The first person 110 also continues holding the right arm 101 of the subject 100 over the shoulder of the left arm 102 while the second person 120 continues holding the legs of the subject 100.

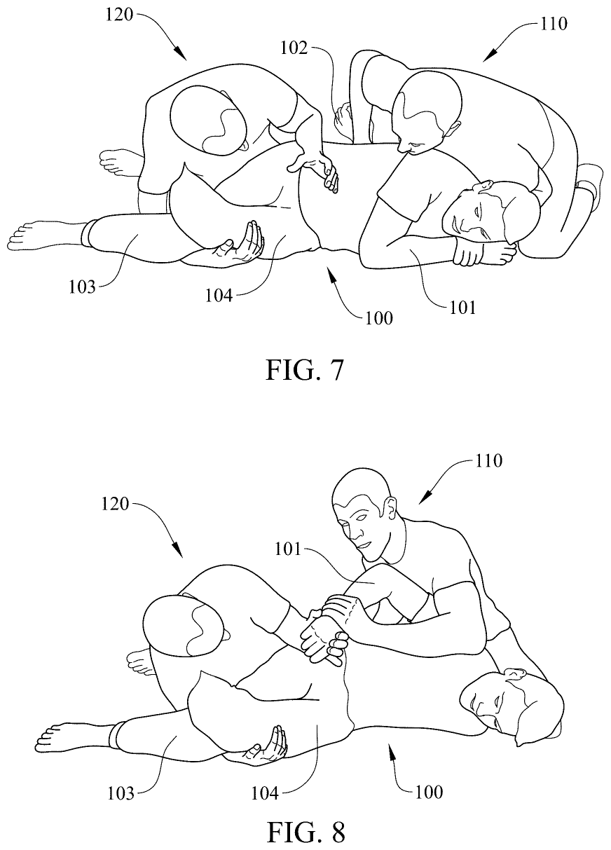

As seen in FIG. 7, the second person 120 may release grip on the left arm 102 and the first person 110 may pull the left arm 102 behind the subject 100. For instance, the first person 110 may pull the left arm 102 of the subject 100 towards himself/herself. Immediately, the first person 110 may push the subject 100 slightly forward and push the lifted head of the subject 100 on the surface where the subject is present (e.g., ground). The elbow on the left arm 102 may thus be lifted from the surface when the left arm 102 is behind the subject 100, i.e., at the back of the subject 100. The first person 110 continues holding the right arm 101 of the subject 100 over the shoulder of the left arm 102 while the second person 120 continues holding the legs of the subject 100. The subject is thus still restrained.

As seen in FIG. 8, the first person 110 may now bring the right arm 101 towards the second person 120 and handover control of the right arm 101 to the second person 120. The first person 110 has one hand holding the left arm 102 behind the subject 100 and with the now free arm after handing over control of the right arm 101, the first person 110 may access handcuffs to be applied to the subject 100.

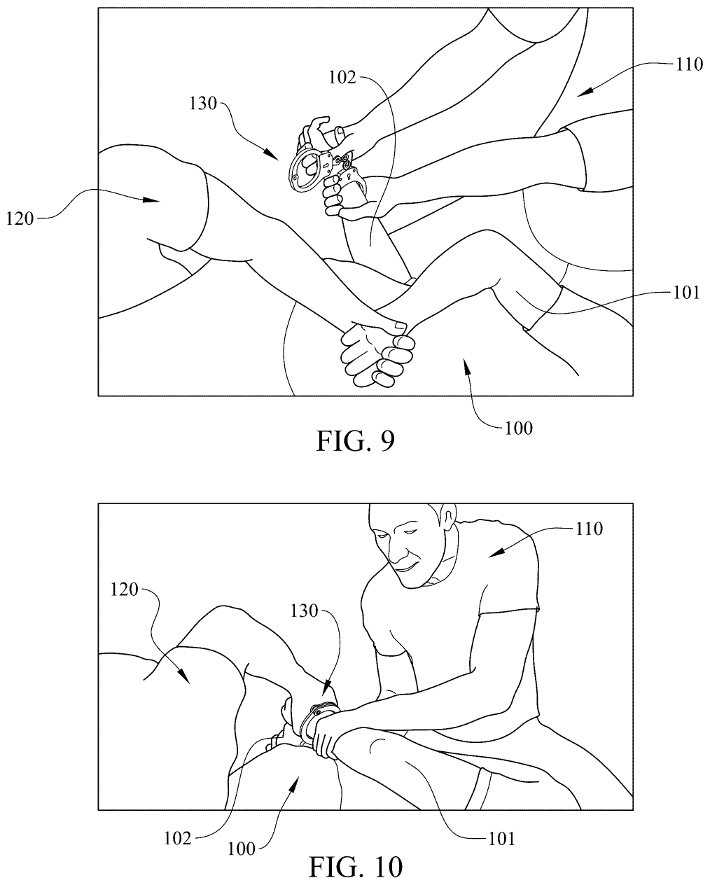

As seen in FIG. 9, the first person 110 may apply handcuffs 130 on the left arm 102 of the subject 100 while the second person 120 continues holding the right arm 101 as well as the legs of the subject 100. As seen in FIG. 10, the second person 120 may now push the right arm 101 behind the subject 100 in order to allow the first person 110 to apply handcuffs 130 on the right arm 101 as well. It is to be noted that although handcuffs are depicted in FIGS. 9-10, the disclosure is not limited thereto and is applicable for other types of restraining devices as well without departing from the scope of the disclosure.

The lateral handcuffing transition described with reference to FIGS. 7-10 is an assisted handcuffing transition, in which, the first person 110 and the second person 120 work together to handcuff the subject 100. In other embodiments, the lateral handcuffing transition may be an unassisted handcuffing transition in which the second person 120 only preserves the cross-over legs position to immobilize the left leg 104 and the right leg 103 while the first person 110 independently works to handcuff the subject 100.

Further, in some embodiments, the left arm 102 may not be gripped by the first person 110 and/or the second person 120 at all times. The left arm 102 may be kept free, however, since the right arm 101 is secured in the cross-over arms position over the shoulder of the left arm 102, the left arm 102 does not interfere in restraining the subject 100. The left arm 102 may thus be the second arm while the right arm 101 is the first arm.

In some embodiments, from the position depicted in FIG. 7, the first person 110 may first handover control of the left arm 102 to the second person while keeping control of the right arm 101. The first person 110 may then access the handcuffs and apply handcuffs on the left arm 102 of the subject 100 while still securing the right arm 101 over the shoulder of the left arm 102. Once the left arm 102 is handcuffed, the first person 110 may take control of the left arm 102 from the second person 120. For instance, the first person 110 may hold the left arm 102 via the handcuffs. Now, the first person 110 may bring the right arm 101 towards the second person 120 and handover control of the right arm 101 to the second person 120. The second person 120 may push the right arm 101 behind the subject 100 in order to allow the first person 110 to apply handcuffs 130 on the right arm 101 as well.

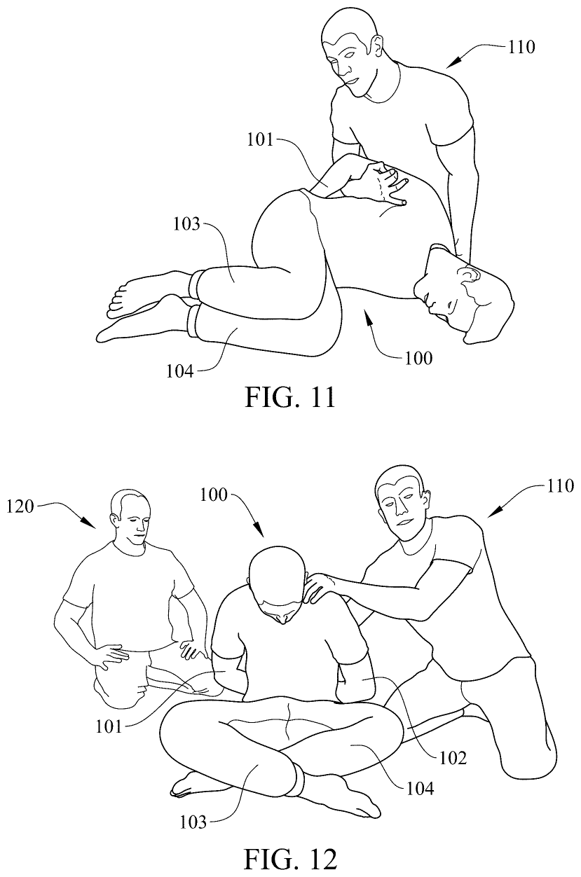

As seen in FIG. 11, the subject 100 with the arms handcuffed is now under control. The second person 120 may release the secure on the legs of the subject 100. That is, the second person 120 may release the left leg 104 which was secured over the right leg 103 of the subject 100. As seen in FIG. 12, the subject 100 may be made to sit and further processes (aftercare, transport, etc.) can be carried out as required.

Accordingly, with the method of lateral restraint described herein, the subject 100 can be restrained without applying pressure on the diaphragm of the subject and without pronating the subject at any stage.

It is to be noted herein that the details will be explained with reference to right and left sides of the subject. In the illustrated embodiments, the subject is shown as lying on the left side and the details have been explained accordingly. Without departing from the scope of the disclosure, the subject may be lying on the right side and in such a scenario, the first arm and first leg may become the second arm and second leg, and vice versa. For instance, the method done on the left leg and left arm may be done on the right leg and right arm, while the method done on the right leg and right arm may be done on the left leg and left arm.

In another aspect, the subject 100 may be in a prone position in which the subject is lying horizontally with the face and torso facing the ground and the back facing upwards. The methods for restraining the subject in the prone position will now be described.

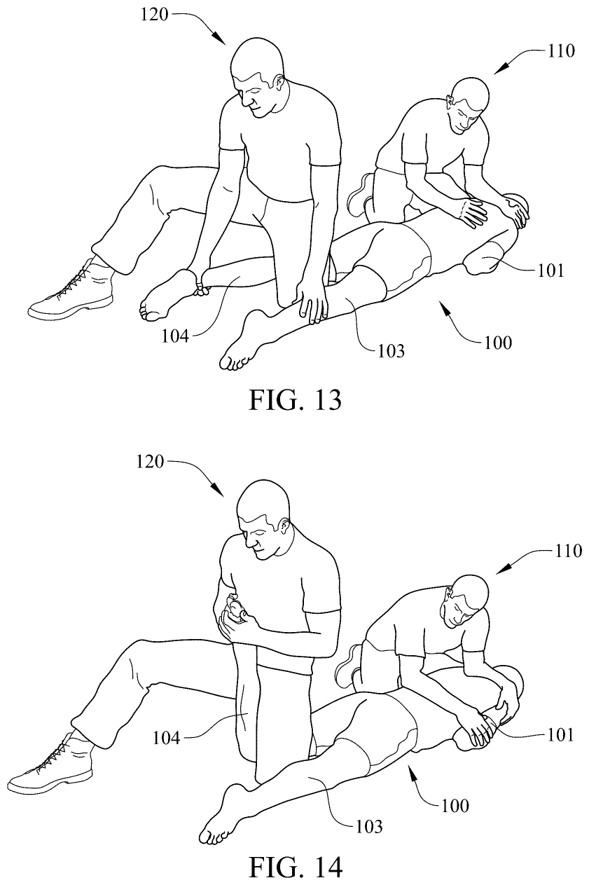

FIGS. 13-20 depict sequential steps of an exemplary method for restraining a subject 100 when the subject is initially in the prone position. The method may be performed by the first person 110 and the second person 120 to shift the subject 100 from the prone position to the lateral restraint position.

Initially, the subject 100 may be in the prone position as depicted in FIG. 13. The subject 100 may be on the ground, on a gurney, a hospital bed, an elevated surface, and the like. The subject 100 may have their hands between the chest and ground to prevent access to the hands. The first person 110 and the second person 120 may position themselves with respect to the subject 100, ensuring full access to the body of the subject 100. The second person 120 may grip the left leg 104 of the subject 100. For instance, the second person 120 may grip the ankle of the left leg 104. The first person 110 may hold the upper body of the subject 100.

As seen in FIG. 14, the second person 120 may bend the knee of the left leg 104 to raise the tibia of the left leg 104. The first person 110 may continue holding the upper body of the subject 100.

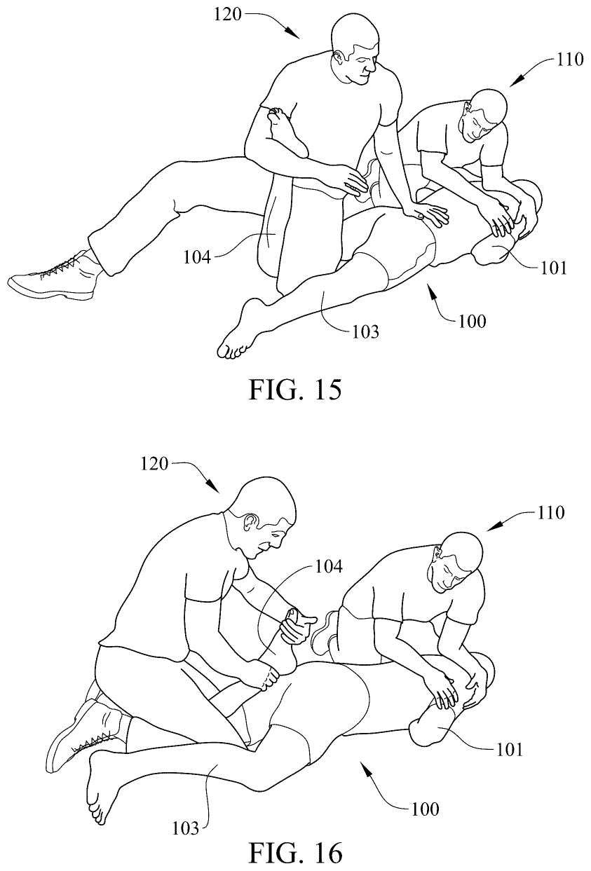

As seen in FIGS. 15-16, the second person 120 brings the tibia of the left leg 104 over the femur and hips of the left leg 104. For instance, the second person 120 may turn towards the first person 110 while securing the tibia of the left leg 104 with one of the arms. Once the second person 120 has turned, the second person 120 may apply a pushing force on the tibia of the left leg 104 to position the tibia over the femur and the hip of the left leg 104. The first person 110 may continue holding the upper body of the subject 100.

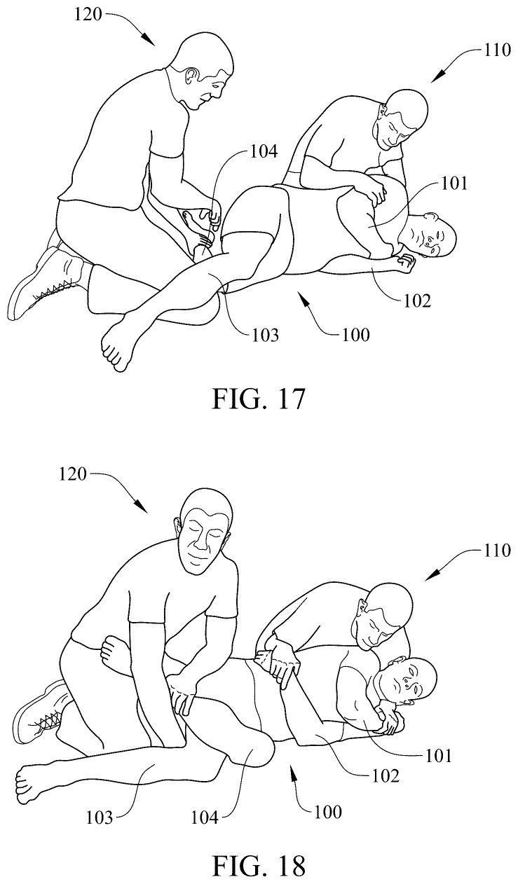

As seen in FIG. 17, the second person 120 may apply a lateral force on the left leg 104 of the subject 100. In the illustrated embodiment, the lateral force may be applied to the left of the subject 100. The lateral force may be applied to rotate the tibia of the left leg 104 in the anti-clockwise direction (considering a point of view of the second person 120 facing the first person 110) towards the ground. The rotation of the tibia of the left leg 104 causes the entire left leg 104, as well as the hips of the subject, to rotate therewith. As depicted, the right leg 103 of the subject 100 has been raised as a result of the rotation of the tibia of the left leg 104 towards the ground, and as a result of the rotation of the hips of the subject 100. Additionally, the torso of the subject 100 is also rotated with the rotation of the hips of the subject 100 such that the hands and torso is exposed to the first person 110. Accordingly, the anti-clockwise rotation of the tibia of the left leg 104 towards the ground forces the subject 100 to shift from the prone position to the lateral position.

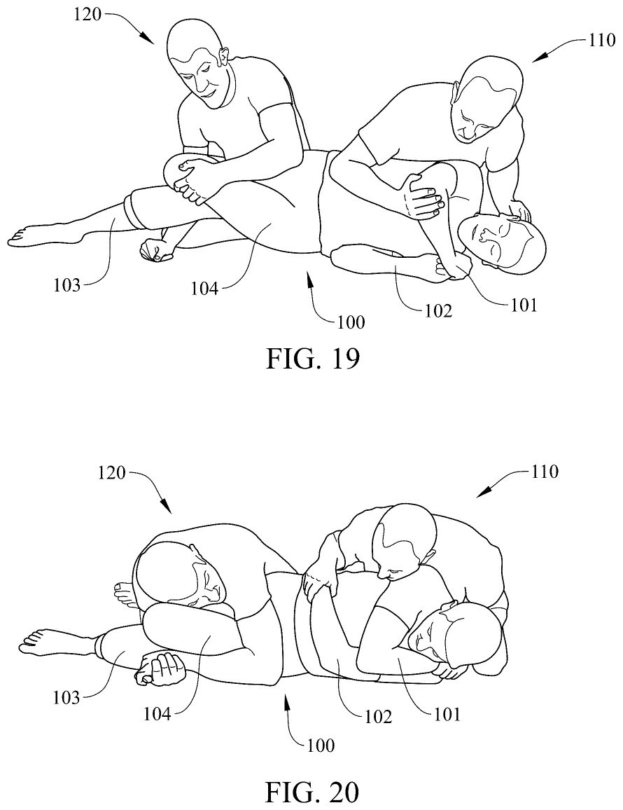

As seen in FIGS. 18-20, once the subject 100 is shifted to the lateral position, the first person 110 may grip the arms of the subject 100 and secure at least the right arm 101 in the cross-over arms position, i.e., the right arm 101 over the shoulder of the left arm 102. The first person 110 may additionally grip the left arm 102 as well. Further, the second person 120 may access the right leg 103 and the left leg 104 of the subject 100. The second person 120 may now position the left leg 104 over the right leg 103 such that the legs are in the cross-over legs position. Once the legs are in the cross-over legs position, the second person 120 may secure the legs in the cross-over legs position. For instance, the second person 120 may raise the legs of the subject 100 as shown in FIG. 19 and insert one of the hand underneath the legs of the subject. The second person 120 may then secure his/her hands to secure the subject 100 in the cross-over legs position. The subject 100 is now restrained in the lateral restraint position similar to the position depicted in FIGS. 3-4. Thereafter, the subject 100 may be handcuffed if required using the handcuffing transition described with reference to FIGS. 5-12 above.

It is to be noted herein that the details have been explained with reference to shifting the subject to the lateral position on the left side of the subject. Without departing from the scope of the disclosure, the subject may be shifted to the lateral position on the right side of the subject. In such a scenario, the method done on the left leg and left arm may be done on the right leg and right arm, while the method done on the right leg and right arm may be done on the left leg and left arm. That is, in such a scenario, the first arm and first leg may become the second arm and second leg, and vice versa. Further, in such a case, the lateral force may be applied to rotate the tibia of the right leg in the clockwise direction towards the ground.

In the embodiments described above, the subject may be an individual who has to be restrained. The first person and the second person may be law enforcement officers, paramedics, firefighters, medical professionals, security personnel, etc.

In another aspect, the subject may be an individual who needs to be completely immobilized and restrained on all the limbs. The subject may be an emotionally disturbed person (EDP) or a patient. The subject may need to be restrained on the ground, on a stretcher, on a bed, and the like.

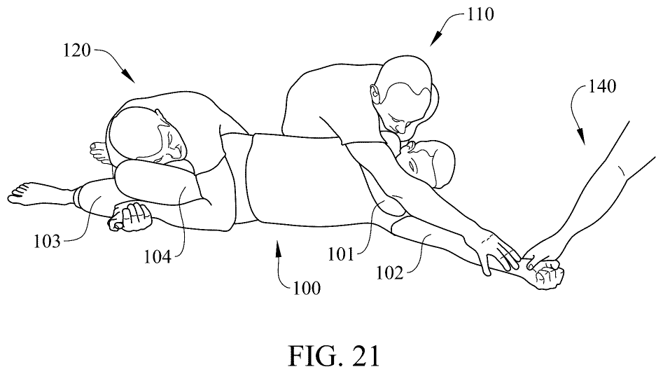

FIGS. 21-25 depict sequential steps of an exemplary method for transitioning from the lateral restraint position to a four-point restraint position. The method may be performed by the first person 110 and the second person 120 shifting the subject 100 from the lateral restraint position to the four-point restraint position. In an embodiment, the subject may be transitioned to the four-point restraint position with assistance from a third person.

Initially, the subject 100 may be in the lateral restraint position on a surface as depicted in FIG. 20 or FIG. 4. In the present scenario, the subject 100 has to be shifted to the four-point restraint position. In an embodiment, assistance from a third person may be required to transition from the lateral restraint position to the four-point restraint position.

As seen in FIG. 21, the second person 120 may continue holding the legs of the subject 100, for instance, by applying a pressure on the legs via the chest of the second person 120. A third person 140 may grip the left arm 102 of the subject 100 while the first person 110 may release grip on the left arm 102. The third person 140, and optionally the first person 110, may pull the left arm 102 so as to extend the left arm 102 of the subject 100. The left arm 102 may be extended outwardly away from the body of the subject 100.

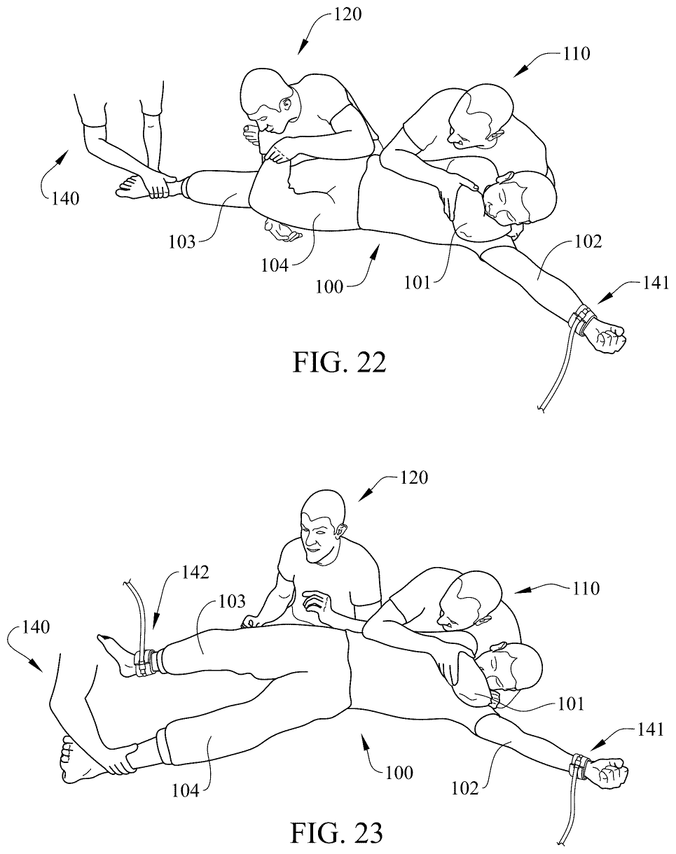

As seen in FIG. 22, the third person 140 may restrain the extended left arm 102 of the subject 100. For instance, the third person 140 may attach a physical restraint 141 on the wrist of the left arm 102, thereby immobilizing the left arm 102. Thereafter, the third person 140 may grip the right leg 103 and straighten the right leg 103. For instance, the third person 140 may grip the right leg 103 via the ankle. The second person 120 may continue restraining the left leg 104 while the first person 110 may continue restraining the right arm 101.

As seen in FIG. 23, the third person 140 may restrain the right leg 103 of the subject 100. For instance, the third person 140 may attach a physical restraint 142 on the ankle of the right leg 103, thereby immobilizing the right leg 103. Thereafter, the third person 140 may grip the left leg 104 and straighten the left leg 104. The second person 120 may handover control of the left leg 104 to the third person 140. Optionally, the second person 120 may assist the third person 140 in straightening the left leg 104. For instance, the third person 140 may grip the left leg 104 via the ankle. Meanwhile, the first person 110 may continue restraining the right arm 101.

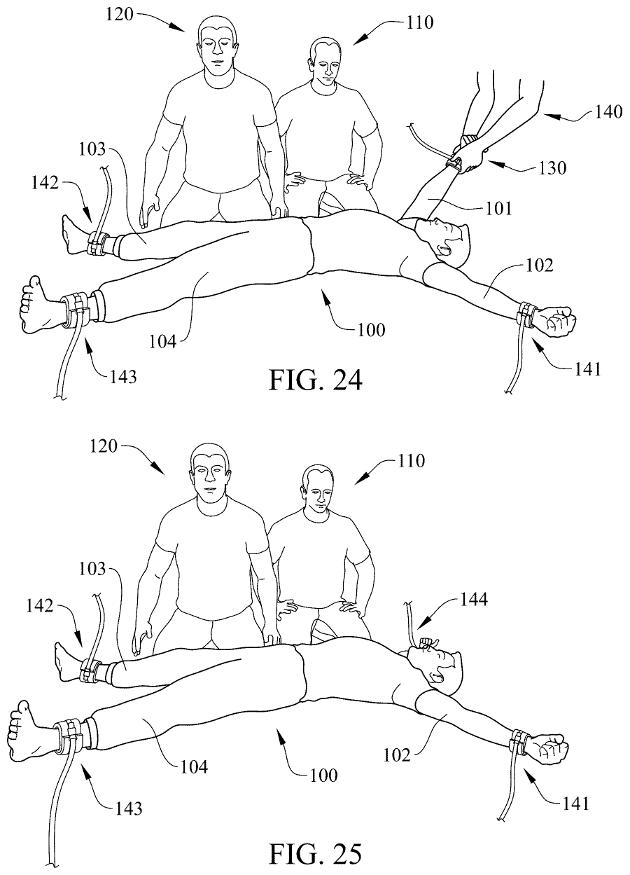

As seen in FIG. 24, the third person 140 may restrain the left leg 104 of the subject 100. For instance, the third person 140 may attach a physical restraint 143 on the ankle of the left leg 104, thereby immobilizing the left leg 104. Thereafter, the third person 140 may grip the right arm 101 while the first person 110 may release grip on the right arm 101. The first person 110 may handover control of the right arm 101 to the third person 140. The third person 140 may pull the right arm 101 so as to extend and straighten the right arm 101. The right arm 101 may be extended outwardly away from the body of the subject 100. The third person 140 may then restrain the extended right arm 101 of the subject 100.

As seen in FIG. 25, the extended right arm 101 of the subject 100 is restrained by a physical restraint 144 in the four-point restraint position. All the limbs of the subject 100 are now restrained by the physical restraints 141, 142, 143, 144. The subject 100 is thus immobilized by restraining of the second arm (left arm 102), followed by the second leg (right leg 103), the first leg (left leg 104), and the first arm (right arm 101). The second limbs are isolated prior to the first limbs. In this order, the subject can be subdued with minimal risk of escape or harm to the restrainers. Moreover, the subject has been transitioned from the prone position to the lateral restraint position, and further to the four-point restraint position, without ever relying on prone controls, thereby reducing the risk of asphyxiation or strangulation of the subject.

It is to be noted herein that the details have been explained with reference to shifting the subject to the lateral position on the left side of the subject and further to the four-point restraint position. Without departing from the scope of the disclosure, the subject may be shifted to the lateral position on the right side of the subject and thereafter to the four-point restraint position. In such a scenario, the method done on the left leg and left arm may be done on the right leg and right arm, while the method done on the right leg and right arm may be done on the left leg and left arm. That is, in such a scenario, the first arm and first leg may become the second arm and second leg, and vice versa. Further, in such a case, the lateral force may be applied to rotate the right leg in the clockwise direction towards the ground.

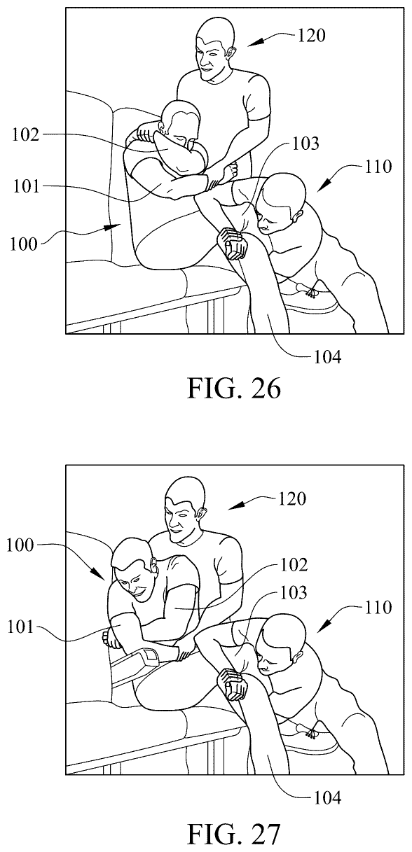

In another aspect, a subject may be seated, and the method may include restraining the seated subject. For instance, the subject may be seated on a chair. FIGS. 26-27 depict sequential steps of an exemplary method for restraining the subject when the subject is seated.

As seen in FIG. 26, when the subject 101 is seated and the restrainers 110, 120 are on the left side of the subject, the first person 110 may direct the subject's left arm 102 over the shoulder of the right arm 101 and secure one or both wrists of the subject 100. The first person 110 may grip the wrists of the subject 100 so as to securely hold both the left arm 102 and the right arm 101 of the subject 100. The second person 120 may cross the subject's right leg 103 over the knee of the left leg 104. A cross-over legs position may be obtained. The second person 120 may further secure the cross-over legs position by extending one arm below the crossed legs of the subject, one arm above the crossed legs of the subject, and secure his/her hands. This configuration prevents the subject from standing or rotating, ensuring the subject remain safely restrained while minimizing any risk of asphyxia or strangulation.

As seen in FIG. 27, the first person 110 may raise the right arm 101 and extend the left arm 102 underneath the lifted right arm 101. The first person 110 may then lower the lifted right arm 101 so as to secure the left arm 102 with the right arm 101. The first person 110 may grip the wrists of the subject 100 so as to immobilize both the left arm 102 and the right arm 101 of the subject 100. Meanwhile, the second person 120 may continue holding the cross-over legs position to immobilize the legs of the subject 100. The subject is thus restrained and there is minimal risk of choking or pressure on vital organs of the subject 100.

Methods and systems for lateral restraint of a subject with minimal risk of compression asphyxia or strangulation are described in the present disclosure. The subject can be shifted from supine position or a prone position to the lateral restraint position in which the subject is laterally restrained. Once the subject is laterally restrained, the subject may be transitioned to the handcuffing position in which the subject can be handcuffed or to the four-point restraint position in which all limbs of the subject are physically restrained.

The described methods are advantageous as compared to conventional methods which often require two or more restrainers lying on top of a single subject, leading to increased risks of compression asphyxia or strangulation resulting from the fact that the subject is restrained in a supine or prone position where the subject is forced to carry the weight of the restrainers directly on their diaphragm or neck. The described methods enable the restrainers to restrain the subject in the medically desirable lateral restraint position for extended periods of time, thereby reducing the danger of compression asphyxia to the subject.

The described methods are crucial for the safe restraint of aggressive, suicidal, or emotionally disturbed individuals. The described methods can be applied across multiple sectors, including:
Law Enforcement: The methods enable officers to safely subdue individuals and transition to handcuffing procedures without prone controls, reducing risks during high-stress encounters.
First Responders: EMTs, paramedics, and firefighters can use the methods to control individuals until law enforcement arrives, increasing safety in volatile situations.
Healthcare: Medical professionals can restrain patients on stretchers with a reduced risk of injury, transitioning to four-point restraints when necessary.
Security: Security personnel in public venues can safely control individuals, reducing the likelihood of injury or panic during restraint.
Education: Educators and school security can use the methods to subdue students who pose a threat, while reducing the risk of injury to themselves and others.

The described methods thus provide a transformative approach to restraint, offering a safer, more effective method that prioritizes the well-being of all involved parties. The advantages of the described methods include:
Reduces Risk of Asphyxia and Strangulation: By maintaining the subject in the lateral position, the methods greatly reduce the risks associated with prone restraints, such as compression asphyxia and strangulation. Positional asphyxia occurs when an individual's physical position prevents them from breathing adequately. Compression asphyxia occurs when external weight is applied to the torso of an individual impairing breathing. When two or more people are using their bodyweight to control the subject in a supine or prone position, the risks of positional asphyxia and compression asphyxia are at their highest, since the subject's ability to expand their lungs easily and naturally is compromised. Using the described methods, the subject is restrained in the medically desirable lateral position such that no external weight is placed on their thoracic region thereby reducing the risk of positional compression asphyxia. Further, when using prone restraints, there is a risk of strangulation when one or both knees of restrainers are positioned on the neck of the subject. When using the described method, at no point is there any pressure placed on or near the vascular structures of the neck of the subject.
Reduces Subject Escape and Weapon Access: The restrainers' control over the subject's arms and legs, combined with the lateral positioning, reduces any escape attempts or access to concealed weapons. During restraint, any escape attempts may result in increased risk for the subject being restrained, the restrainers, and any third parties. The most common and intuitive escape behavior used by individuals subjected to prone restraint, is for them to try to “build the house,” which consist of them pushing off the ground to post on their hands and knees with the final goal of returning to a standing position. Since the described methods provide the restrainers full control of the subject's arms and legs, while preserving the lateral restraint position, the subject is restricted from pronating to “build the house,”. Preventing this escape method, greatly enhances the safety for all parties throughout the encounter. Moreover, when utilizing prone restraint, the subject can reach for their waistband or pocket to retrieve a weapon without detection since their hands are hidden from visibility. When utilizing the lateral restraint position of the described methods, the restrainers have clear visibility and full control of the subject's wrists reducing the subject's ability to access their or someone else's weapon.
Reduces Panic: The lateral restraint position helps to reduce the likelihood of panic, making voluntary compliance more achievable. Being forced into prone restraint, with the weight of two or more people on the subject's body, will often induce panic in the subject. This results in the impaired function of the subject's pre-frontal cortex making voluntary compliance much more challenging to achieve. Since the described method restrains the subject in the lateral position, the likelihood and severity of panic is greatly reduced, making voluntary compliance achievable in the least amount of time possible.
Enhances Safety and Reduces Litigation Risk: The method's effectiveness allows it to be performed by just two people, reducing the need for excessive force, preventing injury, and reducing the risk of restraint-related litigation. Prone restraint is often unreliable and generally requires three, four, five people or more to be effective in what is often characterized as a “dog pile” on the subject. This escalation of force could result in serious injuries or death to the subject. Since the described methods requires only two people to be deployed safely and effectively, it reduces the chance of injury to an absolute minimum. If any people arrive on scene to help, beyond the two applying the system, they can then be utilized to secure the surrounding area, enhancing safety for all involved. Moreover, in every use of force there is a risk of injury, but the escalation of force increases these risks, and the likelihood of excessive force litigation, exponentially. When utilizing the described methods, the effectiveness of the restraint is such that the escalation of force is kept to an absolute minimum. Not only does this reduce the risk of injury to everyone involved, but it also greatly reduces the risk of excessive force litigation.
Facilitates Medical Monitoring and Aftercare: The lateral restraint positioning allows for continuous visual and tactile monitoring of the subject's breathing and overall condition, ensuring that any medical emergencies can be promptly addressed. One of the most significant dangers associated with prone restraint is that once the subject loses consciousness due to asphyxiation, strangulation, or any other medical condition (known or unknown), the restrainers are often unaware of the subject's condition since the subject is lying face-down. Regardless of the cause of the medical emergency, restrainers should continuously monitor the condition of the individual under restraint. The potential undetectability of serious medical events when the subject is in prone restraint is a significant factor for medical emergencies. Due to the lateral body position of the restrained subject in the described methods, the restrainers can visually and tactilely monitor the breathing of the subject, so that immediate aid can be rendered in the event of a medical emergency.
Facilitates Communication and De-Escalation: The described methods facilitate better communication among the restrainers, and between the restrainers and the restrained subject, such as by maintaining eye contact. When prone restraint is used on a subject, it is often chaotic and unpredictable due to the fact that the restrainers are operating independently without a plan, and the random positioning of the restrainers makes communication challenging. When all the members of an organization are aware of the described methods, any two people can work as a team to deploy the system, and communication during the encounter is greatly enhanced by the fact that both restrainers are positioned on one side of the subject so they can make eye contact and calmly speak to one another throughout the entire process. Moreover, one of the core requirements for successful de-escalation is effective communication with the subject. Facial expressions are of critical significance during this process. When the described methods are used, the restrainers have full visibility of the subject's face which greatly facilitates verbal communication while also allowing the restrainers to pick up on the non-verbal cues of the subject.
Facilitates Intervention and Application of Restraint Devices: Law enforcement officers have a “duty to intervene” any time they observe excessive force being used by their colleagues, but this can be very difficult for untrained officers who, themselves, may be panicking. Once a team of two officers arrives trained on the described methods are at the scene of a use of force encounter, not only are they capable of restraining the subject without any additional help, but if they observe untrained officers using excessive force, they can intervene confidently and effectively to keep all parties safe. Moreover, the described methods enable handcuff application on a laterally restrained subject, without any pronation requirement. Instead of handcuff application, the laterally restrained subject can also be safely and securely restrained in four-point restraints.
To summarize, the described methods reduce the risks of: subject asphyxia, subject strangulation, subject escape, subject weapon access, subject panic, subject injury, and excessive force litigation, while facilitating: communication, de-escalation, intervention, application of restraint devices, and aftercare, among other advantages.

Since many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalents.

What is claimed is:

1. A method of restraining a human subject, the method comprising:
manipulating the subject into a restrained position, wherein the restrained position comprises:
a portion of a first arm of the subject over a shoulder of a second arm of the subject to achieve a restrained cross-over arms position; and
a portion of a first leg of the subject over a second leg of the subject to achieve a restrained cross-over legs position, wherein the first arm and the first leg are on opposite sides of a body of the subject;
manipulating the subject to apply a restraining device to the first and second arms of the subject to restrain the first and second arms; and
applying the restraining device to at least one of the first and second arms of the subject.

2. The method according to claim 1 wherein manipulating the subject into the restrained position comprises:
from a supine position, pulling the portion of the first arm of the subject over the shoulder of the second arm of the subject to achieve the cross-over arms position;
rolling the subject from the supine position to a lateral position while preserving the cross-over arms position;
pulling the first leg of the subject over the second leg of the subject to achieve the cross-over legs position; and
while maintaining the cross-over arms position, preserving the cross-over legs position of the subject by securely restraining at least the first leg of the subject.

3. The method according to claim 1 further comprising:
preserving the cross-over arms position by a first restrainer; and
preserving the cross-over legs position by a second restrainer.

4. The method according to claim 3, wherein preserving the cross-over arms position comprises:
securely restraining at least the first arm of the subject by the first restrainer.

5. The method according to claim 1, further comprising:
applying the restraining device to the first and second arms of the subject.

6. The method according to claim 2, wherein preserving the cross-over legs position comprises securely restraining the first leg of the subject by means of one or more of armpits, ribs area, knee area, or by hands of a second restrainer.

7. The method according to claim 2, wherein preserving the cross-over legs position comprises:
raising the legs of the subject by a second restrainer;
extending arms of the second restrainer under the raised legs of the subject; and
securing hands of the second restrainer together to secure the first leg and the second leg in the cross-over legs position.

8. The method according to claim 1, wherein in the cross-over legs position, a tibia of the first leg is crossed over a femur of the second leg.

9. The method according to claim 2, further comprising transitioning the subject to a handcuffing position by:
handing over at least partial control of the second arm from a first restrainer to a second restrainer;
lifting the head of the subject;
pulling the second arm of the subject behind the back of the subject while preserving at least the cross-over legs position;
handing over control of the first arm from the first restrainer to the second restrainer;
applying the restraining device on the second arm of the subject while preserving the cross-over legs position;
pushing the first arm of the subject behind the back of the subject; and
applying the restraining device on the first arm of the subject.

10. The method according to claim 1, wherein when the subject is in the lateral position on its left side, the first arm is the right arm of the subject while the first leg is the left leg of the subject, and
wherein when the subject is in the lateral position on its right side, the first arm is the left arm of the subject while the first leg is the right leg of the subject.

11. The method according to claim 1, further comprising transitioning the subject to a four-point restraint position by:
extending the second arm of the subject and attaching a respective physical restraint to the second arm;
extending the second leg of the subject and attaching a respective physical restraint to the second leg;
extending the first leg of the subject and attaching a respective physical restraint to the first leg; and
extending the first arm of the subject and attaching a respective physical restraint to the first arm.

12. A method of restraining a human subject, the method comprising:
manipulating the subject into a restrained position, wherein the restrained position comprises:
a portion of a first arm of the subject over a shoulder of a second arm of the subject to achieve a restrained cross-over arms position; and
a portion of a first leg of the subject over a second leg of the subject to achieve a restrained cross-over legs position, wherein the first arm and the first leg are on opposite sides of a body of the subject.

13. The method according to claim 12 further comprising:
manipulating the subject to apply a restraining device to the first and second arms of the subject to restrain the first and second arms.

14. The method according to claim 12 wherein manipulating the subject into the restrained position comprises:
from a supine position, pulling the portion of the first arm of the subject over the shoulder of the second arm of the subject to achieve the cross-over arms position;
rolling the subject from the supine position to a lateral position while preserving the cross-over arms position;
pulling the first leg of the subject over the second leg of the subject to achieve the cross-over legs position; and
while maintaining the cross-over arms position, preserving the cross-over legs position of the subject by securely restraining at least the first leg of the subject.

15. The method according to claim 12 further comprising:
preserving the cross-over arms position by a first restrainer; and
preserving the cross-over legs position by a second restrainer.

16. The method according to claim 15, wherein: preserving the cross-over arms position comprises:
securely restraining at least the first arm of the subject by the first restrainer.

17. The method according to claim 15, further comprising securely restraining the second arm of the subject by the first restrainer.

18. The method according to claim 15, wherein preserving the cross-over legs position comprises securely restraining the first leg of the subject by means of one or more of armpits, ribs area, knee area, or by hands of the second restrainer.

19. The method according to claim 15, wherein preserving the cross-over legs position comprises:
raising the legs of the subject by the second restrainer;
extending arms of the second restrainer under the raised legs of the subject; and
securing hands of the second restrainer together to secure the first leg and the second leg in the cross-over legs position.

20. The method according to claim 12, wherein in the cross-over legs position, a tibia of the first leg is crossed over a femur of the second leg.

21. The method according to claim 12, further comprising transitioning the subject to a handcuffing position by:
handing over at least partial control of the second arm from a first restrainer to a second restrainer;
lifting the head of the subject;
pulling the second arm of the subject behind the back of the subject while preserving at least the cross-over legs position;
handing over control of the first arm from the first restrainer to the second restrainer;
applying a restraining device on the second arm of the subject while preserving the cross-over legs position;
pushing the first arm of the subject behind the back of the subject; and
applying the restraining device on the first arm of the subject.

22. The method according to claim 12, wherein when the subject is in the lateral position on its left side, the first arm is the right arm of the subject while the first leg is the left leg of the subject, and
wherein when the subject is in the lateral position on its right side, the first arm is the left arm of the subject while the first leg is the right leg of the subject.

23. The method according to claim 12, further comprising transitioning the subject to a four-point restraint position by:
extending the second arm of the subject and attaching a respective physical restraint to the second arm;
extending the second leg of the subject and attaching a respective physical restraint to the second leg;
extending the first leg of the subject and attaching a respective physical restraint to the first leg; and
extending the first arm of the subject and attaching a respective physical restraint to the first arm.

24. A method for restraint of a human subject in a prone position, the method comprising:
bending a first leg of the subject at the knee so as to raise a tibia of the first leg;
applying a pushing force on the tibia of the first leg to position the tibia over a femur and a hip of the first leg;
rotating the tibia of the first leg towards the ground by applying a lateral force on the first leg, thereby causing rotation of the hips and torso of the subject and shifting the subject to a lateral position;
pulling a first arm of the subject over a shoulder of a second arm of the subject to achieve a cross-over arms position;
preserving the cross-over arms position of the subject by securely restraining at least the first arm of the subject;
pulling the first leg of the subject over a second leg of the subject to achieve a cross-over legs position;
preserving the cross-over legs position of the subject by securely restraining at least the first leg of the subject, wherein the cross-over arms position and the cross-over legs position are preserved simultaneously for restraint of the subject; and
manipulating the subject to apply a restraining device to the first and second arms of the subject to restrain the first and second arms.

25. The method according to claim 24, wherein the cross-over arms position is preserved by a first restrainer and the cross-over legs position is preserved by a second restrainer.

26. The method according to claim 24, wherein when the subject is in the lateral position on its left side, the first arm is the right arm while the first leg is the left leg, and
wherein when the subject is in the lateral position on its right side, the first arm is the left arm while the first leg is the right leg.

27. The method according to claim 25, further comprising transitioning the subject to a restrained position to apply the restraining device, wherein transitioning the subject to the restraining position includes:
handing over at least partial control of the second arm from the first restrainer to the second restrainer;
lifting the head of the subject;
pulling the second arm of the subject behind the back of the subject while preserving at least the cross-over legs position;
handing over control of the first arm from the first restrainer to the second restrainer;
applying the restraining device on the second arm of the subject while preserving the cross-over legs position;
pushing the first arm of the subject behind the back of the subject; and
applying the restraining device on the first arm of the subject.

28. The method according to claim 24, further comprising transitioning the subject to a four-point restraint position in which the subject is restrained all four limbs, wherein transitioning from the lateral restraint to the four-point restraint position comprises restraining the second arm and second leg of the subject prior to the first arm and first leg of the subject.

29. The method according to claim 28, wherein transitioning the subject to the four-point restraint position comprises sequentially restraining the second arm, followed by the second leg, followed by the first leg, and followed by the first arm of the subject.

30. The method according to claim 24, wherein when the subject is shifted in the lateral position on its left side, the tibia of the first leg is rotated in an anti-clockwise direction, and
wherein when the subject is shifted in the lateral position on its right side, the tibia of the first leg is rotated in a clockwise direction.

31. A method for restraint of a human subject in a seated position, the method comprising:
directing a first arm of the subject over a shoulder of a second arm of the subject to achieve a cross-over arms position;
preserving the cross-over arms position by securely restraining the first arm and the second arm of the subject;
crossing a first leg of the subject over a knee of a second leg of the subject to achieve a cross-over legs position; and
preserving the cross-over legs position of the subject, wherein the cross-over arms position and the cross-over legs position are preserved in tandem to restraint the subject in the seated position.

32. The method according to claim 31, further comprising:
raising the second arm of the subject while preserving the cross-over legs position;
extending the first arm underneath the raised second arm of the subject; and
lowering the raised second arm of the subject so as to secure the first arm with the second arm.

33. The method according to claim 31, wherein the cross-over arms position is preserved by a first restrainer and the cross-over legs position is preserved by a second restrainer,
wherein when the first and second restrainers are on a left side of subject, the first arm is the left arm of the subject while the first leg is the right leg of the subject, and
wherein when the first and second restrainers are on a right side of subject, the first arm is the right arm of the subject while the first leg is the left leg of the subject.
